(19) United States
(12) Patent Application Publication
(10) Pub. No.: US 2008/0027423 A1
(43) Pub. Date: Jan. 31, 2008

(54) SYSTEMS FOR TREATMENT OF NASAL TISSUE

(75) Inventors: George Yoseung CHOI, Redwood City, CA (US); Kasey Kai-Chi Li, Palo Alto, CA (US)

Correspondence Address: LEVINE BAGADE HAN LLP, 2483 EAST BAYSHORE ROAD, SUITE 100, PALO ALTO, CA, 94303, US

(73) Assignee: Zoom Therapeutics, Inc., East Palo Alto, CA (US)

(21) Appl. No.: 11/750,862

(22) Filed: May 18, 2007

Related U.S. Application Data
(60) Provisional application No. 60/820,322, filed on Jul. 25, 2006.

(51) Int. Cl. A61N 7/02

(52) U.S. Cl. 606/40

(57) ABSTRACT

Systems for the treatment of nasal tissue, particularly the nasal turbinates, are described. One method for reducing the size of the inferior nasal turbinate is to apply ultrasound energy to the tissue regions beneath the surface of the turbinate tissue. One instrument may be used to deliver ultrasound energy and provide an infusion or injection of a fluid directly into the turbinate being treated, e.g., to bulk up the size of the turbinate to ensure that the ultrasound energy is properly delivered directly into the intended turbinate tissue. Fluids containing anesthetics, fluids infused with analgesics, etc. may be used for pain management while other medications, such as non-steroidal drugs, steroidal drugs, anti-inflammatory drugs, anti-histamines, anti-bacterial drugs, etc., can also be used. Such assemblies can also be utilized with other instruments as a system. For example, such a probe can be used with nasal speculums or imaging instrument in treating tissue.

### CROSS-REFERENCE TO RELATED APPLICATIONS

This application claims the benefit of priority to U.S. Provisional Patent Application No. 60/820,322 filed Jul. 25, 2006 which is incorporated herein by reference in its entirety.

### TECHNICAL FIELD OF THE INVENTION

The present invention relates to devices and methods for clearing obstructed nasal passageways. More particularly, the present invention relates to devices and methods for clearing obstructed nasal tissue by treating the underlying nasal tissues in a safe and efficacious manner by injecting and/or infusion a fluid into the nasal tissues.

### BACKGROUND OF THE INVENTION

Treatments for chronically obstructed airways within the nasal passages of a patient vary greatly. They typically range from the administration of medications to surgical interventional procedures. Examples of typical medication include such types as protriptyline, medroxyprogesterone, acetazolamide, theophylline, nicotine, and other medications. Although helpful at times, they are rarely completely effective. Moreover, such medications frequently have undesirable side effects.

Examples of typical surgical interventions include uvulopalatopharyngoplasty, tonsillectomy, surgery to correct severe retrognathia, and tracheostomy. Other surgical procedures include pulling the tongue as forward as possible and surgically cutting and removing sections of the tongue and other structures which can close off the upper airway passage. These procedures may be effective but the risk of surgery in these patients can be prohibitive and the procedures are often unacceptable to the patients.

As shown in FIG. 1, the sinus cavity 10 which can become obstructed include the nasal passageways leading from the nose 12 to the pharynx 16. The nasal airway has several compartments, namely the inferior 18, middle 20, and superior nasal meatus 22. The turbinates, also referred to as nasal concha, are a series of tissues which form at least a portion of these nasal compartments 18, 20, 22. Forming a portion of the inferior nasal meatus 18 is the inferior nasal turbinate 24. The inferior 24 and middle nasal turbinate 26 each form a portion of the middle nasal meatus 20. The middle 26 and superior nasal turbinate 28 each form a portion of the superior nasal meatus 22. When the inferior 24, middle 26 and/or superior nasal turbinate 28 become enlarged, the various nasal meatus which allow air to pass through the nostril 14 into the pharynx 16 can become obstructed.

Pharmaceuticals such as anti-histamines and anti-inflammatory drugs have been developed for reducing the size of the turbinates. However, pharmaceuticals are not always completely efficacious and generally do not provide a permanent reduction in turbinate size. In addition, pharmaceuticals can have adverse side effects.

Opening of obstructed nasal airways 18, 20, 22 by reducing the size of the turbinates 24, 26, 28 has been performed using surgical and pharmaceutical treatments. Such surgical procedures include anterior and posterior ethmoidectomy, an example of which is a procedure known as the Wigand procedure which involves transecting a portion of the middle turbinate 26. Other procedures have included inserting an electro-surgical probe, such as a radio-frequency (RF) energy probe, directly into a portion of the inferior turbinate 24. Once inserted, RF energy is applied to ablate the tissue interior of the turbinate 24. However, complications, such as excessive hemorrhaging, infection, perforation, scarring, adhesion of the turbinate, and intra-operative and post-operative pain may be present.

Accordingly, there exists a need for devices and methods which are efficacious and safe in clearing obstructed nasal passageways, at least for an extended period of time.

### SUMMARY OF THE INVENTION

By reducing the size of a nasal turbinate, particularly the inferior nasal turbinate, obstruction of a nasal meatus such as the inferior nasal meatus can be reduced thereby improving the air flow through the nasal meatus. One method for reducing the size of the inferior nasal turbinate involves applying ultrasound energy to the tissue regions beneath the surface of the inferior turbinate. Ultrasound energy may be particularly advantageous in damaging the tissues beneath the turbinate surface layer by enabling the delivery of energy to a predetermined distance through the tissue without damaging the tissue surface while injuring the underlying tissue to create scarring. Moreover, because ultrasound energy may leave the turbinate tissue surface undisturbed, the need for surgical cutting is obviated.

One variation of a treatment instrument which may be used to deliver ultrasound energy to the underlying turbinate tissue may also be configured to provide an infusion or injection of a fluid directly into the turbinate being treated by the ultrasound energy. The fluid injected into the turbinate may be used to bulk up the physical size of the turbinate by injecting the fluid to present a larger surface area to the ultrasound transducers positioned along the instrument. The enlarged surface area may help to ensure that the ultrasound energy is properly delivered directly into the intended turbinate tissue rather than surrounding tissues.

The injected fluid may also be used for drug delivery directly into the treated turbinate tissue. For instance, anesthetic fluids or other fluids infused with analgesics may be injected into the turbinate tissue to provide for pain management during and after the application of the ultrasound energy. Additionally, other drugs for injection may include any number of medications, such as non-steroidal drugs, anti-inflammatory drugs, anti-bacterial drugs, etc. which may be injected to control excessive post-operative swelling as well as infection. Additionally, the one or more injection needles may be utilized as a positioning tool for ensuring that the ultrasound energy, which is directional, is delivered into the intended turbinate tissue. For example, the injection needle(s) may be initially positioned directly within the turbinate tissue prior to application of the ultrasound energy since the ultrasound transducer(s) along the probe may be aligned with the injection needle(s). Accordingly, if the needle(s) is positioned directly within the turbinate tissue to be treated, the operator may be assured that the ultrasound energy will be directionally aligned with the appropriate turbinate tissue region.

The ultrasound and infusion probe may have an elongate shaft which is sufficient to allow for insertion and advancement into the nasal cavity and against the appropriate turbinate tissue surface. The distal end portion may be angled relative to the elongate shaft or it may be straight depending upon the desired configuration. The distal end portion may have an end effector assembly which has one or more hollow infusion/injection needles which are retractably disposed within the distal end portion. During advancement into the nasal cavity and positioning against the turbinate tissue, the infusion/injection needles may be positioned within the distal end portion so as to present a smooth atraumatic surface to the tissue. When a fluid is to be injected into the tissue after the probe has been desirably positioned against the tissue surface, a control or advancement mechanism on handle, which is connected to a proximal end of the shaft, may be actuated to advance the needles at least partially out of the distal end portion. Between or adjacent to the needles are one or more ultrasound transducers along the body of the distal end portion.

An electronic/fluid cable is electrically and fluidly connected to the handle and is further connected to a power/infusion assembly, which may hold a fluid reservoir and a pump electrically coupled to a controller or central processor. Any of the above-mentioned fluids, e.g., analgesics, anesthetics, anti-inflammatory drugs, water, saline, etc., may be filled within the reservoir for delivery through the cable and through the one or more infusion/injection needles for delivery into the turbinate tissue.

In use, the elongate shaft and distal end portion may be advanced through the patient's nostril and through the inferior nasal meatus against the tissue surface of the inferior nasal turbinate. The distal end portion of the elongate shaft may be positioned anywhere against the inferior nasal turbinate and the infusion/injection needles may be deployed from the distal end portion and pierced into the turbinate tissue, where the fluid may be injected and/or infused from the needles into the turbinate. As the fluid is injected into the tissue, the infused inferior turbinate may begin to expand in size thereby pressing against the distal end portion. The fluid may be stopped and the focused ultrasound energy may then be transmitted from the transducers into the underlying expanded turbinate tissue.

Once the injection and ultrasound treatment has been concluded, the damaged underlying turbinate tissue may scar and eventually reduce a size of the inferior turbinate, thereby resulting in an unobstructed inferior nasal meatus. The treatments may be performed periodically between extended time periods while the turbinate tissue regenerates or on an as-needed basis.

In alternative configurations, the distal end effectors may include a mechanism for securely pressing the surface of the elongate shaft against the turbinate tissue surface to be treated to ensure piercing of the needles into the tissue as well as sufficient contact for the ultrasound transmission. For instance, expandable balloons and wires or ribbon members which may be reconfigured from a low-profile configuration against the elongate shaft to an expanded shape may be utilized.

Moreover, the ultrasound and infusion probe may optionally include an additional radio-frequency energy generator to deliver RF energy to one or more needles to ablate the pierced tissue. The ultrasound and infusion probe may also optionally include a cooling unit fluidly connected via a fluid line to the power/infusion assembly. Cooled fluid may be fluidly connected through the elongate shaft to a cooling fluid port positioned along the distal end portion.

Additionally, the probe may be utilized in a system with any number of instruments. For instance, the probe may be integrated with a nasal speculum for facilitate entry and placement within the patient's nasal cavity. Moreover, the probe and/or nasal speculum may additionally be utilized with a number of different attachment mechanisms for facilitating procedures while under visualization.

### BRIEF DESCRIPTION OF THE DRAWINGS

FIG. 1 shows an illustrative view of a nasal cavity and the passageways formed by the turbinates.

FIG. 2 shows a variation of a treatment instrument which may be used to deliver ultrasound energy as well as for providing an infusion or injection of a fluid directly into the turbinate being treated by the ultrasound energy.

FIGS. 3A and 3B illustrate partial cross-sectional detail views of a distal end portion of the elongate shaft showing the infusion/injection needles positioned within and projected out from the elongate shaft, respectively.

FIG. 3C shows another variation of an elongate shaft having an infusion/injection needle retractably positioned on a distal end of the shaft and with one or more ultrasound transducers also on the distal end.

FIG. 3D shows another variation of an elongate shaft having an additional tissue engaging tip on the distal end.

FIG. 3E shows yet another variation of an elongate shaft having a combination of infusion/injection needles along the length and distal end of the device.

FIG. 4A shows an elongate shaft advanced through the inferior nasal meatus for treating the inferior nasal turbinate.

FIG. 4B shows alternative positions for placing the elongate shaft against the turbinate to be treated.

FIG. 4C shows an elongate shaft advanced through the nostril for treating an anterior portion of the inferior nasal turbinate.

FIGS. 5A to 5C illustrate one method for infusing or injecting the fluid into the inferior turbinate and applying ultrasound energy to the expanded tissue and the resulting unobstructed inferior nasal meatus.

FIG. 6 illustrates an alternative variation where a single needle may be utilized with one or two ultrasound transducers.

FIG. 7 illustrates yet another alternative variation where three or more needles may be utilized with at least two ultrasound transducers in an alternating manner.

FIGS. 8A and 8B show variations for positioning of the needles and transducers relative to one another.

FIGS. 9A to 9C show side and end views, respectively, of one variation of a distal end portion which may be configured to include an expandable balloon.

FIGS. 10A and 10B show side and end views, respectively, of another variation of a distal end portion which may be configured to include a reconfigurable wire or ribbon member.

FIG. 11 shows an alternative configuration of the ultrasound and infusion assembly which may optionally utilize an RF generator and/or an optional cooling fluid reservoir assembly.

FIGS. 12A and 12B show top and side views, respectively, of an alternative ultrasound and infusion probe which may be configured to have a plurality of ultrasound transducers.

FIGS. 13A and 13B show side views of examples of an elongate shaft which is malleable or has at least a malleable portion.

FIG. 14 illustrates an alternative variation for utilizing cooling or cryo-therapy for anesthetizing the tissue prior to or during treatment.

FIG. 15 shows another variation of utilizing cooled or chilled fluid for anesthetizing the tissue.

FIG. 16 shows yet another variation of anesthetizing the tissue utilizing thermo-electric cells for cooling the underlying tissue.

FIGS. 17A and 17B show perspective and end views, respectively, of an assembly utilizing an elongate probe integrated with a nasal speculum to facilitate advancement of the instrument into the patient's nasal cavity.

FIG. 18 shows an example of a coupling mechanism which may be positioned between the retraction members of a nasal speculum and allows for translational and pivotal movement of the retained shaft relative to the speculum body.

FIGS. 19A and 19B show an assembly and exploded assembly view, respectively, of an integrated treatment and visualization assembly.

FIG. 20 shows a perspective view of an attachment which may be temporarily connected to a rhinoscope.

FIG. 21 shows a partial cross-sectional view of one example of the attachment.

FIG. 22 shows a partial cross-sectional view of another example of the attachment utilizing an integrated probe as a single attachment to a rhinoscope.

FIG. 23 shows yet another example in the partial cross-sectional view of an attachment assembly utilizing an automatic controller and/or motor assembly.

### DETAILED DESCRIPTION OF THE INVENTION

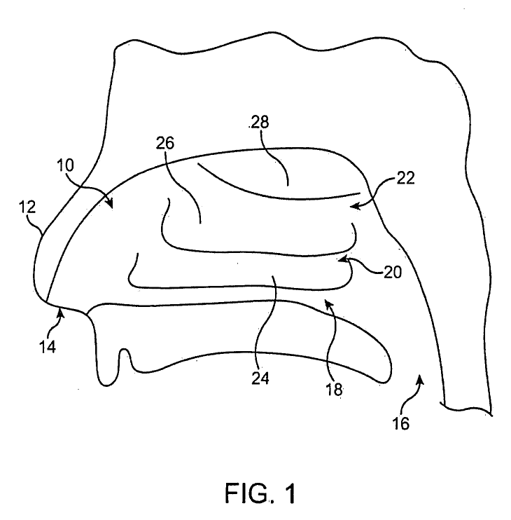

As described above in FIG. 1, connecting the nostril 14 and pharynx 16 are the passageways of the inferior nasal meatus 18, the middle nasal meatus 20, and the superior nasal meatus 22. Forming at least a portion of each of these passageways are the nasal turbinates. Forming at least a portion of the inferior nasal meatus 18 is the inferior nasal turbinate 24. Forming at least a portion of the middle nasal meatus 20 is the inferior nasal turbinate 24 and the middle nasal turbinate 26. Forming at least a portion of the superior nasal meatus 22 is the middle nasal turbinate 26 and the superior nasal turbinate 28.

By reducing the size of a nasal turbinate, particularly the inferior nasal turbinate 24, obstruction of a nasal meatus such as the inferior nasal meatus 18 can be reduced. By reducing an obstruction of a nasal meatus, air flow through the nasal meatus is improved. One method for reducing the size of the inferior nasal turbinate 24 involves the application of ultrasound energy to the tissue regions beneath the surface of the inferior turbinate 24. Ultrasound energy may be particularly advantageous in damaging the tissues beneath the turbinate surface layer by enabling the delivery of energy to a predetermined distance through the tissue without damaging the tissue surface while injuring the underlying tissue to create scarring. Moreover, because ultrasound energy may leave the turbinate tissue surface undisturbed, the need for surgical cutting is obviated. The affected targeted tissue may scar and atrophy and eventually shrink and/or prevent the enlargement of the turbinate 24.

Although reference is made particularly to treatment of the inferior turbinate 24, this is done so for illustrative purposes. The procedures and devices described herein may easily be applied to any of the nasal turbinates 24, 26, 28 and are intended to be so.

However, because the size of the turbinate to be treated may vary greatly between patients, there is variability in the application of ultrasound energy that an ultrasound energy delivery device needs to compensate for. Additionally, even the application of ultrasound energy may produce pain and discomfort in the patient being treated due to the highly vascularized structure of the turbinates.

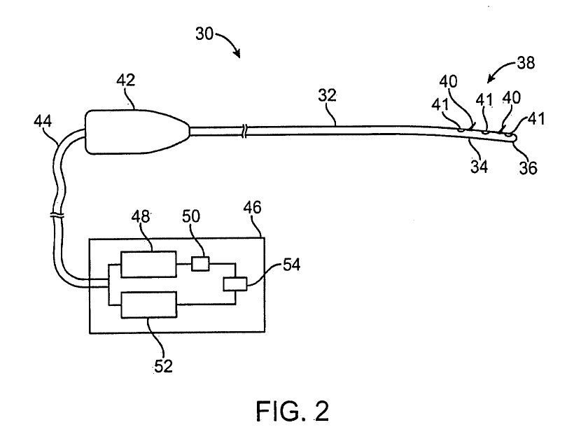

FIG. 2 illustrates a variation of a treatment instrument which may be used to deliver ultrasound energy for treating the tissues underlying the turbinate surface as well as for providing an infusion or injection of a fluid directly into the turbinate being treated by the ultrasound energy. The fluid injected into the turbinate may serve a number of different purposes. One purpose is to bulk up the physical size of the turbinate by injecting the fluid to present a larger surface area to the ultrasound transducers positioned along the instrument. The enlarged surface area may help to ensure that the ultrasound energy is properly delivered directly into the intended turbinate tissue rather than surrounding tissues. Examples of fluids which may be used for bulking the turbinate tissue may include any number of suitable fluids, e.g., saline, water, etc.

Another purpose is for drug delivery directly into the treated turbinate tissue. For instance, anesthetic fluids or other fluids infused with analgesics (e.g., lidocaine with or without epinephrine, marcaine with or without epinephrine, etc.) may be injected into the turbinate tissue to provide for pain management during and after the application of the ultrasound energy. Additionally, other drugs for injection may include any number of medications, such as steroidal drugs (e.g., corticosteroids, dexamethasone, beclomethasone, etc.), non-steroidal drugs (e.g., non-steroidal anti-inflammatory drugs, etc.), anti-inflammatory drugs, anti-histamines (e.g., diphenhydramine, etc.), anti-bacterial drugs, etc. which may be injected to control excessive post-operative swelling as well as infection.

Yet another purpose may be to utilize the one or more injection needles as a positioning tool for ensuring that the ultrasound energy, which is directional, is delivered into the intended turbinate tissue. For example, the injection needle(s) may be initially positioned directly within the turbinate tissue prior to application of the ultrasound energy since the ultrasound transducer(s) along the probe may be aligned with the injection needle(s). Accordingly, if the needle(s) is positioned directly within the turbinate tissue to be treated, the operator may be assured that the ultrasound energy will be directionally aligned with the appropriate turbinate tissue region.

Returning now to FIG. 2, ultrasound and infusion probe 30 is illustrated as having an elongate shaft 32 with a distal end portion 34 having a rounded or blunted atraumatic tip 36 to prevent trauma to contacted tissue. Elongate shaft 32 may have a length which is sufficient to enable the insertion of distal end portion 34 into the nasal cavity of a patient. Accordingly, the length of shaft 32 may range anywhere from several centimeters to 25 cm or longer while the distal end portion may range anywhere, e.g., from 10 to 30 mm in length or longer if so desired. The elongate shaft 32 itself may have conform to any cross-sectional area so long as the overall size is sufficient to allow for insertion and advancement into the nasal cavity and against the appropriate turbinate tissue surface. However, elongate shaft 32 may be typically circular with a diameter ranging anywhere from 4 to 5 mm or more. Moreover, elongate shaft 32 may optionally define one or more visual markings or indicators along its length to indicate a depth of the shaft 32 into the nasal cavity by comparison against the patient nostril 14.

The distal end portion 34 may be angled relative to the elongate shaft 32 or it may be straight depending upon the desired configuration. The distal end portion 34 may have an end effector assembly 38 which has one or more hollow infusion/injection needles 40 which are retractably disposed within the distal end portion 34. During advancement into the nasal cavity and positioning against the turbinate tissue, the infusion/injection needles 40 may be positioned within the distal end portion 34 so as to present a smooth atraumatic surface to the tissue. When a fluid is to be injected into the tissue after the probe 30 has been desirably positioned against the tissue surface, a control or advancement mechanism on handle 42, which is connected to a proximal end of shaft 32, may be actuated to advance needles 40 at least partially out of distal end portion 34.

The illustration of FIG. 2 shows two retractable infusion/injection needles 40; however, fewer or additional needles 40 may be utilized depending upon the desired results and procedure to be undertaken. Between or adjacent to needles 40 are positioned, one or more ultrasound transducers 41 along the body of distal end portion 34. The illustration shows three ultrasound transducers for delivering the ultrasound energy, but fewer or additional transducers 41 may be utilized or positioned along the distal end portion 34.

An electronic/fluid cable 44 is electrically and fluidly connected to handle 42 and is further connected to a power/infusion assembly 46. Within assembly 46 is a fluid reservoir 48 and a pump 50 electrically coupled to controller or central processor 54. Any of the above-mentioned fluids, e.g., analgesics, anesthetics, anti-inflammatory drugs, water, saline, etc., may be filled within reservoir 48 for delivery through cable 44, elongate shaft 32 and through the one or more infusion/injection needles 40 for delivery into the turbinate tissue. The infusion rate of the fluid and control of the pump 50 may be determined by the controller 54. An example of a pump which is pre-programmed to inject a fluid in a controlled injection rate and which may be utilized with the pump 50 is commercially available as the CompuDent® delivery system and Wand® handpiece (Milestone Scientific, Inc., South Orange Livingston, N.J.). Power supply 52 may also be provided within assembly 46 and may be controlled by controller 54 to control the amount of energy provided by the ultrasound transducers 41 located in distal end portion 34.

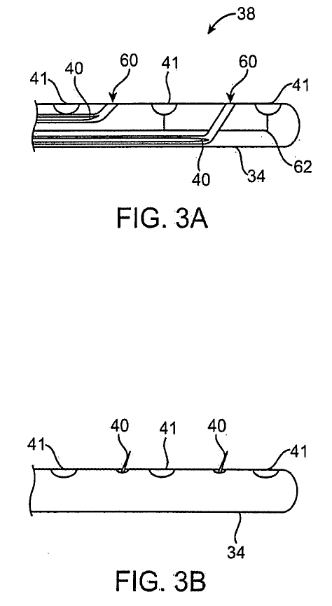

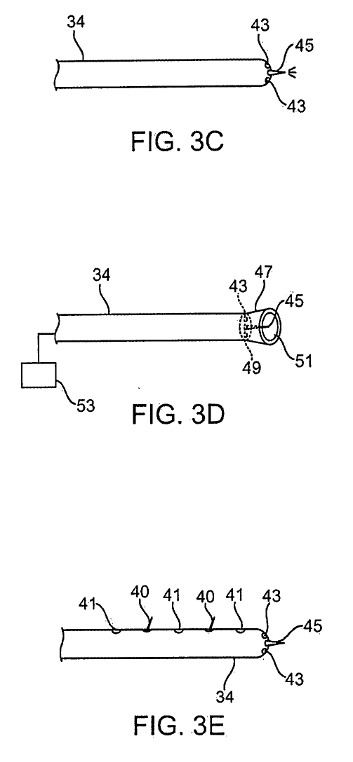

As mentioned above, during delivery and positioning of elongate shaft 32 against the turbinate tissue, the one or more needles 40 may be retracted within distal end portion 34, as shown in the partial cross-sectional detail view of FIG. 3A. As illustrated, infusion/injection needles 40 may be positioned within their respective needle lumens 60 positioned between the ultrasound transducers 41. The piezoelectric transducers of each of the ultrasound transducers 41 may be electrically coupled via wires 62 routed through elongate shaft 32 to the power supply 52 located within assembly 46. The piezoelectric transducer may be vibrated over a range of frequencies, e.g., anywhere from 0.5 to 12 MHz, or more typically between 5 to 12 MHz, to generate the ultrasound energy to treat the turbinate tissue.

When the infusion/injection needles 40 are to be deployed into or against the turbinate tissue, they may be advanced distally through needle lumens 60 until they project from a surface of the elongate shaft 32, as shown in FIG. 3B. Needles 40 may be configured to project from shaft 32 from less than 1 mm to more than 2 mm or anywhere therebetween provided that needles 40 are able to sufficiently contact against and/or into the turbinate tissue surface to inject the fluid.

In another variation, FIG. 3C illustrates the distal end portion 34 of elongate shaft 32 having at least one infusion/injection needle 45 retractably disposed at the distal tip. Also located at the tip are one or more ultrasound transducers 43 positioned adjacent to the retractable needle 45. Such a variation may be particularly useful for treating anterior portions of turbinate tissue.

FIG. 3D shows yet another variation in which the distal tip of distal end portion 34 further includes a tissue engaging hood 47 protruding distally from shaft 32. Hood 47 may be a removable or integrated tapered structure defining an opening 51 in communication with a vacuum lumen 49, which may be in fluid communication with a vacuum pump 53. Retractable needle 45 may be deployable to project into and/or through the opening 51 for contacting any turbinate tissue engaged therewith. In use, tissue engaging hood 47 may be positioned proximate or adjacent to a tissue region to be treated and a vacuum force through lumen 49 may be activated to securely draw the tissue therein. Once the drawn-in tissue is secured within engaging hood 47 by the vacuum, needle 45 may be projected into the secured tissue for injecting any fluids for treatment. Moreover, one or more ultrasound transducers 43 may also be positioned within the opening 51 to further treat the vacuum-secured tissue via ultrasound energy, as described herein. Once treatment has been completed, the vacuum may be de-activated to disengage the tissue.

FIG. 3E shows yet another variation which combines the ultrasound transducers 41 and retractable infusion/injection needles 40 which project along the length of the elongate shaft 32, as shown in FIG. 3B, with the distally disposed ultrasound transducers 43 and retractable infusion/injection needle 45 which projects from the distal tip of the shaft. This particular variation may be utilized to treat all aspects of the turbinate tissue, including the anterior and lateral portions of the tissue.

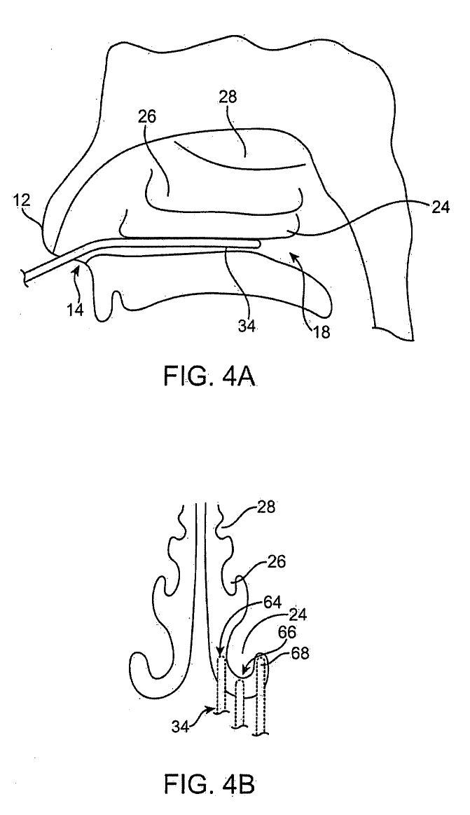

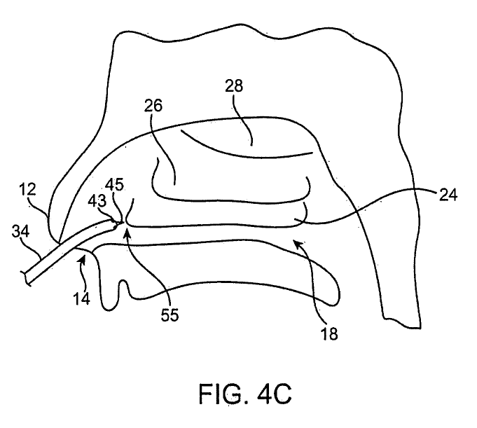

In use, elongate shaft 32 and distal end portion 34 may be advanced through the patient's nostril 14 and through the inferior nasal meatus 18 against the tissue surface of the inferior nasal turbinate 24, as shown in FIG. 4A. Distal end portion 34 of elongate shaft 32 may be positioned anywhere against the inferior nasal turbinate 24 at a first lateral surface 64, against an inferior surface 66, at a second lateral surface 68, or any or all three positions of the inferior turbinate 24, as shown in the end view of the turbinates 24, 26, 28 in FIG. 4B.

The instrument variations shown and described above in FIGS. 3C to 3E may be utilized in particular for treating anterior portions of the turbinate tissues, as previously mentioned. As illustrated in FIG. 4C, the distal portion 34 of the shaft 32 may be advanced through the patient's nostril 14 and positioned adjacent to an anterior portion 55 of the turbinate tissue. The infusion/injection needle 45, which may optionally be retracted during advancement into the nasal cavity or fully deployed, may inserted into the anterior portion 55 to inject the fluids. During and after the injection of fluids, the one or more ultrasound transducers 43 may be activated on the distal tip of the shaft to treat the underlying tissue, as further described below. Once the treatment has been completed, the shaft may be removed or repositioned to another portion of tissue for treatment.

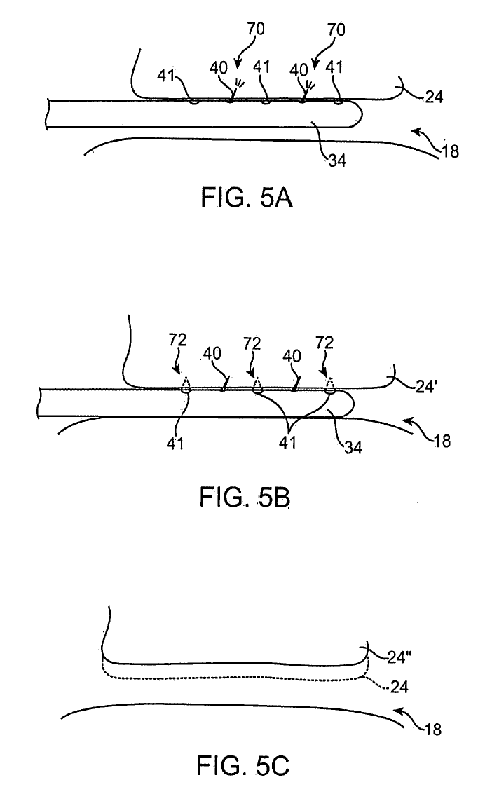

As described above and as illustrated in FIG. 5A, the infusion/injection needles 40 may be deployed from distal end portion 34 and pierced into the turbinate tissue 24, where the fluid 70 may be injected and/or infused from needles 40 into the turbinate 24. As the fluid is injected into the tissue, the infused inferior turbinate 24′ may begin to expand in size, as shown in FIG. 5B, thereby pressing against distal end portion 34. The fluid may be stopped and the focused ultrasound energy 72 may then be transmitted from transducers 41 into the underlying expanded turbinate tissue 24′. The ultrasound energy 72 may be applied anywhere from 1 second to 1 minute, and more particularly anywhere from 2 to 45 seconds and can be fired sequentially or simultaneously. Moreover, the focal point of the ultrasound energy 72 may range anywhere from about 1 mm or more away from the transducers 41 and more particularly anywhere from 2 to 4 mm away, so long as the focal point of the ultrasound energy 72 is able to be focused into the underlying turbinate tissue 24′ leaving the turbinate tissue surface unperturbed.

The increased size of the turbinate 24′ tissue surface presented to the transducers 41 may facilitate treatment of the underlying tissue as well as ensure that the appropriate tissue is treated. Moreover, once the ultrasound energy 72 has been applied at a first location, the needles 40 may be retracted and the distal end portion 34 may be moved to another region of the inferior turbinate 24′ to further effect treatment. Any amount of the expanded inferior turbinate 24′ may be treated, e.g., 3 to 4 cm of turbinate tissue along its length. With the infusion of anesthetics and/or anti-inflammatory drugs, any pain associated with the application of ultrasound energy and scarring of the tissue is eliminated or reduced.

Once the injection and ultrasound treatment has been concluded, the damaged underlying turbinate tissue may scar and eventually reduce a size of the inferior turbinate 24″, thereby resulting in an unobstructed inferior nasal meatus 18, as shown in FIG. 5C. The treatments may be performed periodically between extended time periods while the turbinate tissue 25″ regenerates or on an as-needed basis.

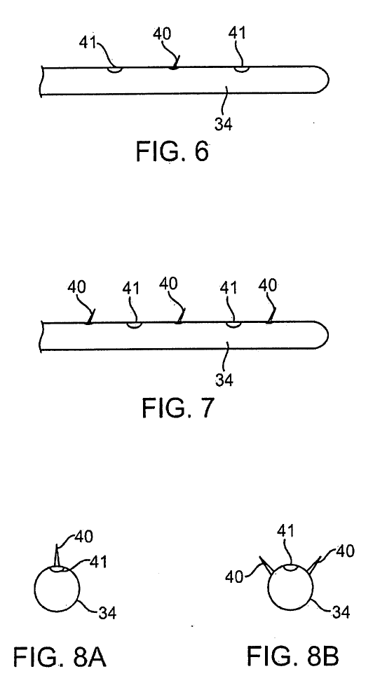

The configuration and number of infusion/injection needles 40 and ultrasound transducers 41 may be varied depending upon the desired effect. FIG. 6 illustrates an alternative variation where a single needle 40 may be utilized with one or two ultrasound transducers 41. Alternatively, FIG. 7 shows a variation where three or more needles 40 may be utilized with at least two ultrasound transducers 41 in an alternating manner. Moreover, the circumferential positioning of the needles 40 relative to the transducers 41 may also be varied. FIG. 8A shows one variation where each of the needles 40 and transducers 41 may be aligned linearly while FIG. 8B shows another variation where two or more needles 40 may be off-set to project at an angle relative to one another with the ultrasound transducer 41 positioned therebetween.

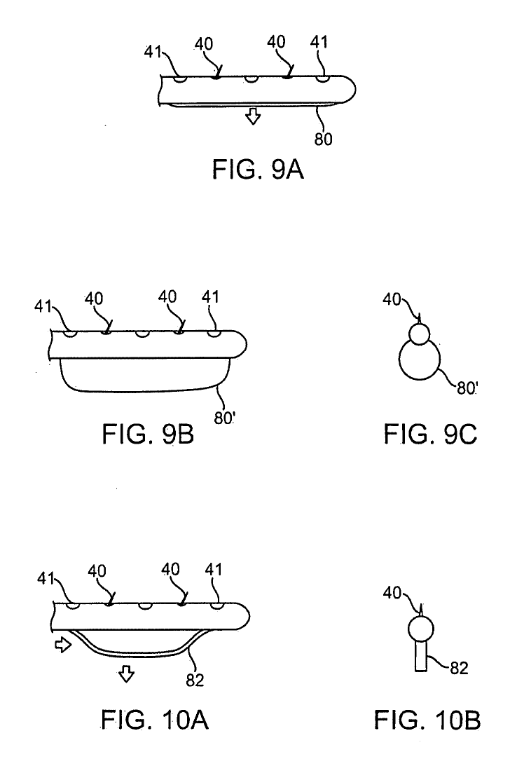

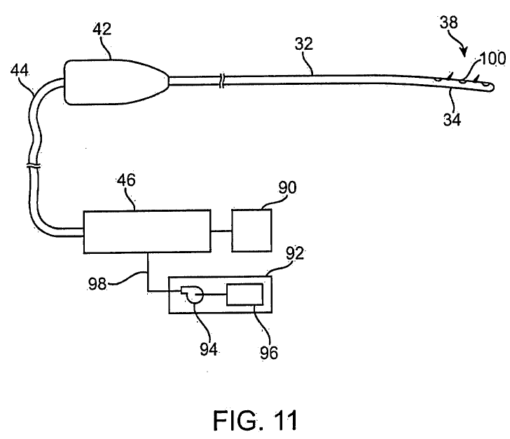

In alternative configurations, the distal end effectors may include a mechanism for securely pressing the surface of the elongate shaft against the turbinate tissue surface to be treated to ensure piercing of the needles into the tissue as well as sufficient contact for the ultrasound transmission. For instance, FIG. 9A illustrates one variation of a distal end portion which may be configured to include an expandable balloon 80. Once the shaft has been desirably positioned against the turbinate tissue surface, balloon 80′ may be expanded via a fluid such as water or saline or a gas such as air delivered through an inflation lumen defined through shaft 32, as shown in FIG. 9B and the end view in FIG. 9C. The expanded balloon 80′ may be utilized to press against the surrounding tissue within the inferior nasal meatus 18 to directionally press or force the shaft surface and needle 40 against or into the turbinate tissue. Once the desired treatment has been completed, balloon 80′ may be deflated and the elongate shaft 32 may be moved to another region of the turbinate or removed entirely.

Another variation of a mechanism is shown in the side and end views of FIGS. 10A and 10B, which illustrate a wire or ribbon member 82 which may be reconfigured from a low-profile configuration against the elongate shaft 32 to an expanded shape, as shown. When the elongate shaft 32 is to be securely presented against the tissue surface, wire or ribbon member 82 may be advanced or actuated from handle 42 to urge the member 82 into a reconfigured and expanded shape to push against the tissue.

In yet another configuration, the ultrasound and infusion probe 30 may optionally include an additional radio-frequency energy generator 90, which may be configured to deliver RF energy to one or more needles to ablate the pierced tissue. Ablation of the pierced regions of tissue may help to coagulate the pierced tissue. Moreover, the ultrasound and infusion probe 30 may also optionally include a cooling unit 92 fluidly connected via fluid line 98 to power/infusion assembly 46. Cooling unit 92 may comprise a pump 94 fluidly coupled to a reservoir 96 containing cooled or chilled fluid 96, e.g., saline, water, etc. The cooled fluid 96 may be fluidly connected through elongate shaft 32 to a cooling fluid port 100 positioned along distal end portion 100. Before, during, or after ultrasound energy transmission into the turbinate tissue, the cooled fluid may be pumped from reservoir 96 through cooling fluid port 100 to cool the surface of the turbinate tissue to ensure that the turbinate tissue surface is unperturbed by the energy applied beneath its surface.

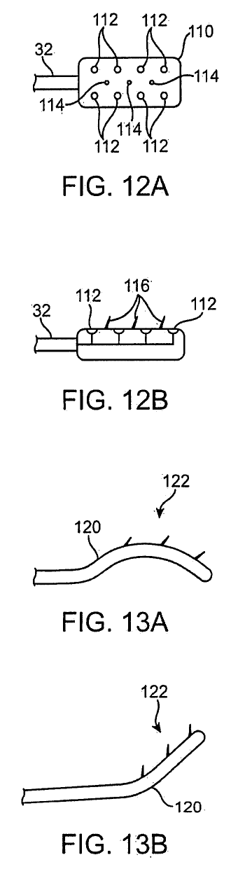

Other configurations for the ultrasound and infusion probe may be utilized. One example is shown in the top and side views of the ultrasound and infusion probe 110 shown in FIGS. 12A and 12B, respectively. In this configuration, a plurality of ultrasound transducers 112 may be positioned over a surface of the probe 110 and one or more needle openings 114 may be similarly positioned over the surface adjacent to the transducers 110. An example of a probe having multiple ultrasound transducers is shown in further detail in U.S. Pat. No. 6,361,531 to Hissong, which is incorporated herein by reference in its entirety. The one or more infusion/injection needles 116 may be deployed through the openings 114 when pressed against the turbinate tissue surface.

In any of the variations described herein, elongate shaft may be configured to be a malleable shaft 120, or at least have a distal portion which is malleable, from which the one or more infusion/injection needles 122 may be positioned. Such a malleable shaft may be configured by the user to conform to any number of configurations prior to advancement into the nasal cavity. For instance, the malleable shaft 120 may be configured into a curved configuration, as shown in FIG. 13A, or an angled configuration, as shown in FIG. 13B. In either case, once the procedure has been performed, the malleable shaft 120 may be reconfigured into yet another shape depending upon the desired configuration and anatomy of the patient.

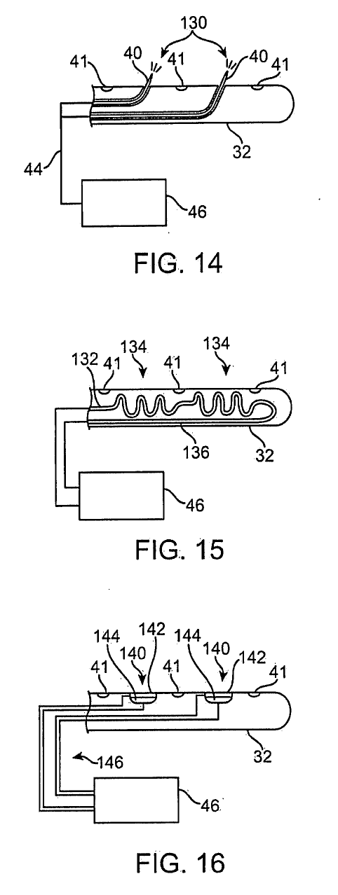

Aside from injecting fluids, such as water or saline, or anesthetic fluids or other fluids infused with analgesics into the underlying tissue, as described above, any of these fluids may be chilled or cooled prior to injection into the tissue to facilitate the anesthetizing of the tissue prior to ultrasound treatment. Turning now to FIG. 14, an alternative variation for utilizing cooling or cryo-therapy is shown where the fluid 130 injected into the tissue may be cooled or chilled via a cooling unit in assembly 46 prior to injection. Moreover, because the cooled fluid 130 facilitates anesthetization, anesthetics or analgesics may optionally be omitted from the fluid altogether. Moreover, the cooled fluid 130 may also be squirted or sprayed from the needles 40 onto the tissue surface prior to insertion of the needles 40 to dull any pain which may be associated with entry into the tissue.

In another variation for utilizing chilled or cooled fluid for anesthetization of the underlying tissue, FIG. 15 shows another variation where a cooling line may be routed through the probe 32. A feed line 132 carrying a cooled fluid may be routed through the probe 32 and coiled or looped or otherwise configured to optimize heat exchange in one or more heat exchange regions 134 along the shaft surface such that any tissue which contacts directly, or is in proximity to, any one of the heat exchange regions 134 may be cooled by the flowing chilled fluid flowing through feed line 132. The spent cooling fluid may be returned to assembly 46 by return line 136, where it may be recharged by a cooling unit in the assembly 46.

In yet another variation, FIG. 16 shows an example utilizing one or more thermoelectric cooling cells 140, e.g., Peltier cells, which may be positioned along the shaft surface adjacent to or in-between the one or more ultrasound transducers 41. The cooling cells 140 may be connected and powered via corresponding electrical wires 146 to assembly 46, which may contain the power supply and/or controller for controlling the cooling of cells 140. The thermo-electric cooling cells 140 may be positioned along the probe surface such that junction 142 is cooled along the outer surface of probe 32 while the portion of junction 144, which is conversely heated, faces within probe 32 and away from the tissue surface. Heated junction 142 may be in contact with a conductor or cooling line within probe 32 to maintain a temperature of probe 32 against the underlying tissue.

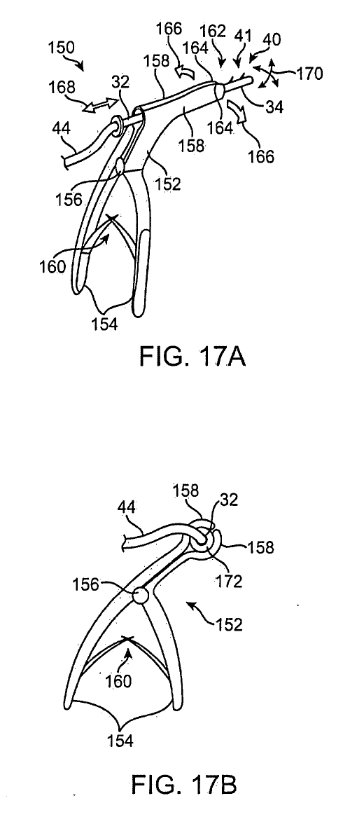

Although the ultrasound and infusion probe assembly may be utilized alone in treating the turbinate tissues, it may also be utilized with a device or mechanism for maintaining an opening of the nasal passage to facilitate treatment, such as a nasal speculum. In one example, nasal speculum treatment assembly 150 is shown in the perspective view of FIG. 17A in which the elongate probe shaft 32 may be integrated with a nasal speculum body 152 such that shaft 32 is translationally positioned between the nasal retraction members 158 such that when retraction members 158 are used to spread the nostrils of a patient's nose, shaft 32 may be advanced directly through the nostril relative to speculum body 152 along probe guide channel 162 and directly into position against or adjacent to the turbinate tissue for treatment.

Speculum handles 154 may be articulated by the user to position nasal retraction members 158, which may pivot relative to one another via hinge or pivot 156, to retract the tissue surrounding the patient's nostril. Arrows 166 illustrate the movement of retraction members 158 to spread the nasal tissue. Once the desired positioning has been determined, handle lock 160 may be actuated to maintain a position of the handles 154 and thus maintain a position of the retraction members 158 and nasal tissue.

With the nostrils retracted and the turbinate tissue exposed, the probe shaft 32 may be advanced distally relative to the speculum body 152, as described above and as illustrated by arrow 168. Moreover, the probe shaft 32 may not only be translationally held between retraction members 158 but also pivotably relative to retraction members 158, as indicated by arrows 170, such that distal end portion 34 of shaft 32 with the needles 40 and ultrasound transducers 41 may be positioned proximate to or directly against varying tissue anatomy.

To further facilitate treatment, speculum retraction members 158 may be provided with one or more lighting elements 164 to illuminate the tissue area. Such lighting elements 164 may utilize any number of configurations and lighting mechanisms, e.g., light emitting diodes, fluorescence, chemiluminescence, incandescent lighting, etc.

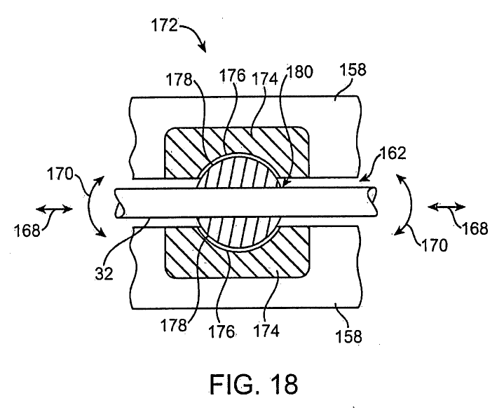

FIG. 17B illustrates an end view of the speculum body 152 and probe shaft 32 coupled thereto via a shaft coupling mechanism 172. FIG. 18 illustrates an example of a coupling mechanism 172 which may be positioned between retraction members 158 and allows for translational and pivotal movement of the retained shaft 32 relative to the speculum body 152. In this example, coupling member 174 is shown in a partial cross-sectional top view retained between retraction members 158 defining socket joint 176. A ball joint member 178, which may retainingly surround a portion of probe shaft 32 within shaft engagement portion 180, may be pivotably and receiving retained within socket joint 176, as indicated by arrows 170. The coupling mechanism 172 itself may be slidingly retained within a groove or channel defined along a length of retraction members 158 to allow for translation of the assembly therealong, as indicated by the arrows 168.

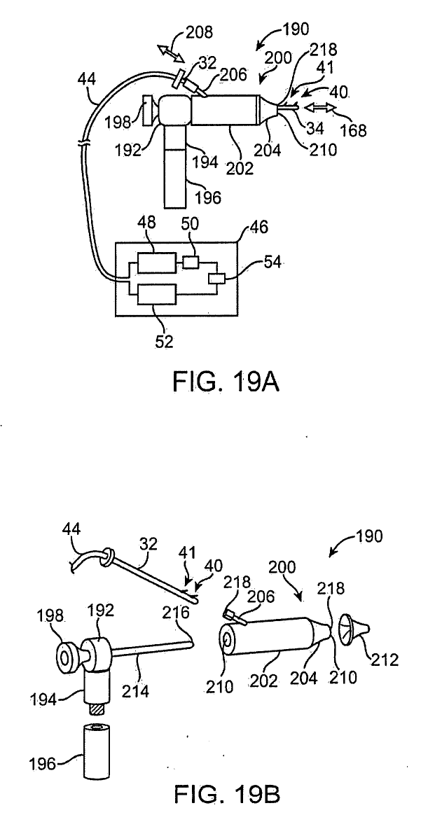

In yet another variation, the treatment probe assembly may be integrated as a removable attachment 200 to a conventional oto-endoscope or rhinoscope 192 to form an integrated treatment and visualization assembly 190, as illustrated in the assembly view of FIG. 19A. As shown in this example, rhinoscope 192 may have handle 194 attachable to a power supply and lighting source 196 as well as an eyepiece 198, which is connectable to an image processing unit for displaying on a monitor, through which tissue regions of interest may be visualized via the rhinoscope 192. The removable attachment 200 may comprise an attachment body 202 slidably disposed over the rhinoscope 192 and having a speculum or tapered distal portion 204 for insertion through a nostril of a patient.

Attachment 200 may further have an access port 206 extending at an angle from the attachment 200 through which the probe shaft 32 may be slidably positioned or disposed. Probe shaft 32 may be slidably disposed through access port 206 and through shaft lumen 218 such that the end effector distal end portion 34 may be maintained with needles 40 in their retracted state within attachment 200 prior to and/or during insertion of tapered insertion portion 204 through the patient's nostril. Then under direct visualization of rhinoscope 192, probe shaft 32 may be urged translationally 208 such that distal end portion 34 projects distally from shaft lumen 218 and into contact against or proximate to the appropriate tissue to be treated, where the needles 40 may be urged to project from the shaft surface and into the tissue for treatment, as described above.

FIG. 19B shows an exploded assembly view of the treatment and visualization assembly 190. As illustrated, probe shaft 32 may be removably insertable within access port 206 and shaft lumen 208 of attachment 202. Also shown is rhinoscope 192 with rhinoscope shaft 214 removably insertable within attachment 202 through rhinoscope lumen 210. Attachment 202 is configured to have a length such that the distal imaging and lighting tip 216 of rhinoscope 192 is positionable proximal to, coincident with, or just beyond a distal opening of rhinoscope lumen 210 to allow for sufficient visualization of the tissue to be treated when insertion portion 204 is positioned within the patient. A disposable covering 212 may also be removably placed over the tapered insertion portion 204 to allow for use between different patients.

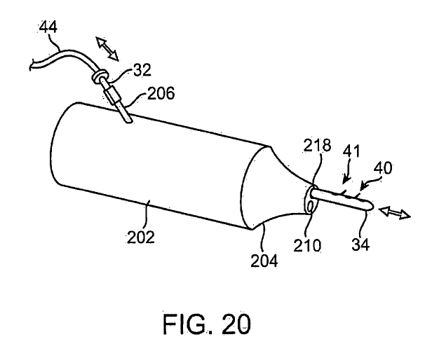

FIG. 20 shows a perspective view of attachment 202 illustrating one example of how the openings for shaft lumen 218 and rhinoscope lumen 210 may be positioned relative to one another. In other variations, additional lumens and corresponding access ports may be included through attachment 202 to allow for the use of additional instruments for other procedures, as desired.

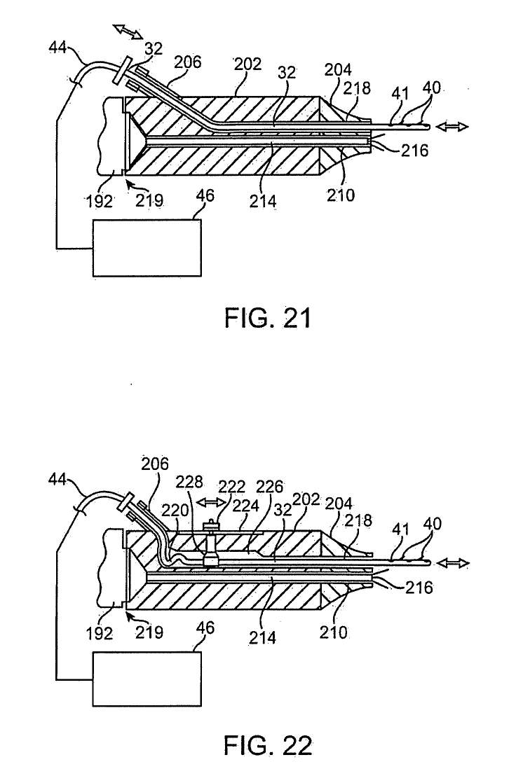

FIG. 21 shows a partial cross-sectional view of one example of attachment 202 illustrating the positioning of probe shaft 32 through angled access port 206 and shaft lumen 218. Also shown is rhinoscope shaft 214 positioned within rhinoscope lumen 210 such that the imaging and lighting tip 216 is positioned near or at the distal end of insertion portion 204. Attachment 202 may be removably coupled for temporary engagement via any number of engagement mechanisms 219 (e.g., interference fit, engaging detent, locking projections, etc.) to the base portion of rhinoscope 192.

In yet another variation, FIG. 22 illustrates a probe shaft 32 integrated with attachment 202 as a single attachment to rhinoscope 192. In this variation, probe shaft 32 may have its proximal end affixed to shaft attachment 228 housed within lumen 226 which is defined through attachment 202. Cable 44, which is in communication with power supply and/or infusion assembly 46 may be coupled to shaft attachment 228 via a flexible looped section 220 within attachment 202. The shaft attachment 228 itself may be connected to a control mechanism 222, such as a manipulatable slide mechanism or control, that the user may articulate to slide along control channel or groove 224 to translate the probe shaft 32 proximally and distally relative to attachment 202 and the tissue being treated. Furthermore, in such a variation the probe shaft 32 may be optionally detachable from shaft attachment 228 such that replacement probe shafts 32 may be used. Alternatively, the entire attachment assembly 202 may be disposable thereby allowing for replacement attachments 202 to be used with the rhinoscope 192.

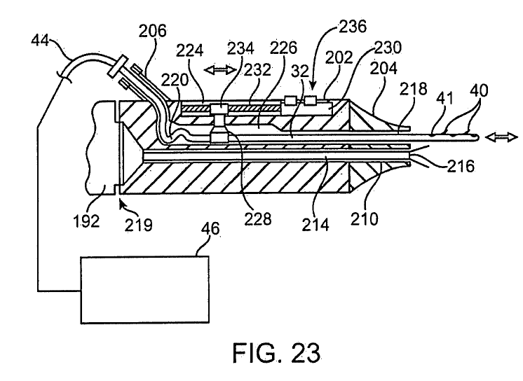

Another variation is illustrated in the partial cross-sectional view of FIG. 23, which shows an attachment assembly similar to that above but having an automatic controller and/or motor assembly. A controller and/or motor assembly 230 may be optionally integrated within attachment 202 and connected to a mechanism for translating the probe shaft 32, e.g., turn screw 232, over which a threaded carriage 234 may coupled. The carriage 234 may be connected to shaft attachment 228, which in turn may be connected to a proximal end of probe shaft 32. In use, controller and/or motor assembly 230 may be activated by the user to rotate turn screw 232 in a proximal and/or distal direction. As turn screw 232 is rotated in the appropriate direction, the carriage 234 may be urged to translate linearly along turn screw 232 through the housing of attachment 202 in a corresponding direction, thereby moving shaft probe 32 linearly either proximally into attachment 202 or distally to extend at least partially from attachment 202.

One or more control elements 236 may be located along an outer surface of attachment 202 to actuate the controller and/or motor assembly 230. Moreover, a controller which may be located within assembly 230 may be configured to automatically advance probe 32 distally a predetermined distance into the patient's nasal cavity while under visualization from the imaging tip 216 of rhinoscope 192. Additionally, the assembly may also be configured to communicate with assembly 46 and not only automatically advance the injection needles 40 into underlying tissue and inject the appropriate fluids, but to also actuate the one or more ultrasound transducers 41 into the injected tissue. Moreover, after the treatment has been completed, the controller in assembly 230 may also be configured to not only retract the injection needles 40 but to also retract probe shaft 32, as well as any other functions as desired.

Upon completion of the procedure upon the patient, attachment 202 may be removed from rhinoscope 192 and from assembly 46 and sterilized before use upon another patient or simply disposed.

The applications of the devices and methods discussed above are not limited to the treatment of the nasal turbinates but may include any number of further treatment applications. Other treatment sites may include areas or regions of the body such as soft tissue bodies. Modification of the above-described assemblies and methods for carrying out the invention, and variations of aspects of the invention that are obvious to those of skill in the art are intended to be within the scope of the claims.

What is claimed is:

1. A system for treating tissues within a nasal cavity, comprising:
an elongate shaft having a distal end, a proximal end, and a length having at least one ultrasound transducer positioned near or at the distal end and at least one needle disposed near or at the distal end, wherein the at least one needle is retractably positioned to extend from a surface of the shaft; and
a speculum having an insertion portion sized to be inserted at least partially within a nostril, wherein the speculum further defines an opening sized for passage of the elongate shaft therethrough into the nostril.

2. The system of claim 1 wherein the elongate shaft is sized to be advanced through the nostril and into a nasal meatus of the nasal cavity.

3. The system of claim 1 wherein the elongate shaft is malleable.

4. The system of claim 1 wherein the at least one ultrasound transducer is positioned adjacent to the at least one needle.

5. The system of claim 1 further comprising a plurality of ultrasound transducers positioned near or at the distal end.

6. The system of claim 1 wherein the at least one ultrasound transducer has a focal point of at least 1 mm.

7. The system of claim 1 wherein the at least one needle comprises a hollow infusion or injection needle.

8. The system of claim 1 further comprising a plurality of needles disposed near or at the distal end, wherein the plurality of needles are retractably positioned to extend from the surface of the shaft.

9. The system of claim 1 further comprising a handle assembly attached to the proximal end of the elongate shaft.

10. The system of claim 1 further comprising a fluid reservoir in fluid communication with the at least one needle.

11. The system of claim 1 further comprising a power supply in electrical communication with the at least one ultrasound transducer.

12. The system of claim 1 further comprising an expandable member disposed near or at the distal end, wherein the expandable member is reconfigurable to an expanded configuration which urges the at least one needle against a tissue region of interest.

13. The system of claim 1 further comprising a cooling fluid reservoir in fluid communication with at least one cooling port defined near or at the distal end.

14. The system of claim 1 wherein the speculum comprises a nasal speculum.

15. The system of claim 1 wherein the elongate shaft is translationally movable with respect to the speculum.

16. The system of claim 15 wherein the elongate shaft is pivotably movable with respect to the speculum.

17. The system of claim 1 further comprising a housing through which the elongate shaft is advanceable and the speculum comprises a tapered distal portion of the housing.

18. The system of claim 17 further comprising a disposable covering adapted to be fitted over the tapered distal portion of the housing.

19. The system of claim 17 further comprising a rhinoscope positionable adjacent to the elongate shaft through the tapered distal portion of the housing.

20. The system of claim 17 further comprising a control mechanism translatable along the housing and connected to at least a portion of the elongate shaft.

21. The system of claim 17 further comprising a motor coupled to at least a portion of the elongate shaft.

22. The system of claim 21 further comprising a controller coupled to at least a portion of the elongate shaft.

23. A method of treating tissue within a nasal cavity, comprising:
retracting tissue surrounding a nostril to facilitate entry therethrough;
advancing an elongate shaft through the retracted nostril such that a distal end of the shaft is positioned against or proximate to a tissue region of interest within the nasal cavity;
piercing the tissue region via at least one needle retractably disposed near or at the distal end;
infusing or injecting a fluid through the at least one needle into the tissue region; and
applying ultrasound energy beneath a surface of the tissue region via at least one ultrasound transducer positioned near or at the distal end.

24. The method of claim 23 wherein retracting comprises retracting the tissue via a nasal speculum.

25. The method of claim 23 wherein retracting comprises retracting the tissue via a tapered distal portion.

26. The method of claim 23 wherein advancing comprises advancing the elongate shaft through an inferior nasal meatus.

27. The method of claim 26 further comprising contacting the distal end against an inferior nasal turbinate.

28. The method of claim 23 wherein piercing comprises piercing the tissue region via a plurality of needles.

29. The method of claim 23 wherein piercing further comprises advancing the at least one needle from within the elongate shaft to project externally of a surface of the elongate shaft.

30. The method of claim 23 wherein infusing or injecting comprises infusing or injecting a fluid selected from the group consisting of anesthetics, analgesics, anti-inflammatory drugs, anti-histamines, non-steroidal drugs, steroidal drugs, anti-bacterial drugs, water, and saline.

31. The method of claim 23 wherein applying comprises transmitting ultrasound energy at least 1 mm away from the at least one ultrasound transducer.

32. The method of claim 23 wherein applying comprises transmitting ultrasound energy via a plurality of ultrasound transducers positioned near or at the distal end.

33. The method of claim 23 further comprising applying a cooling fluid onto the surface of the tissue region.

34. The method of claim 23 further comprising urging the distal end against the tissue region of interest prior to applying ultrasound energy beneath a surface.

35. The method of claim 23 wherein advancing comprises automatically advancing the elongate shaft via a motor.

36. The method of claim 23 wherein piercing comprises automatically piercing the tissue via a controller.

37. The method of claim 23 wherein infusing or injection comprises automatically infusing or injecting the fluid via a controller.

38. The method of claim 23 wherein applying comprises applying ultrasound energy via a controller.

39. The method of claim 23 further comprising visualizing the tissue via a rhinoscope.
